(19) United States
(12) Patent Application Publication
(10) Pub. No.: US 2005/0033145 A1
(43) Pub. Date: Feb. 10, 2005

(54) Wearable tissue viability diagnostic unit

(76) Inventors: John S. Graham, Bel Air, MD (US); Dominic P. Bua, Lynnfield, MA (US); Kevin T. Schomacker, Maynard, MA (US)

Correspondence Address: OFFICE OF THE STAFF JUDGE ADVOCATE;U.S. ARMY MEDICAL RESEARCH AND MATERIEL COMMAND, ATTN: MCMR-JA (MS. ELIZABETH ARWINE), 504 SCOTT STREET, FORT DETRICK, MD, 21702-5012, US

(21) Appl. No.: 10/882,310

(22) Filed: Jul. 2, 2004

Related U.S. Application Data
(60) Provisional application No. 60/484,784, filed on Jul. 2, 2003.

(51) Int. Cl. A61B 5/05

(52) U.S. Cl. 600/407; 600/476

(57) ABSTRACT

A device for gathering image information about a region of tissue that has been exposed to a contrast agent and methods of use thereof. The device preferably includes night vision goggles, and an excitation source that generates light of a wavelength to activate the contrast agent. The excitation source preferably is attached to the night vision goggles and is capable of directing light to a target. A filter preferably is attached to the night vision goggles, wherein the filter passes light sufficient to form an image of the region of tissue, and wherein the image may be assessed to determine the viability of the region of tissue.

### II. CROSS-REFERENCE TO RELATED APPLICATION

This patent application claims the benefit of U.S. Provisional Patent Application No. 60/484,784, filed Jul. 2, 2003, which is hereby incorporated by reference.

### I. STATEMENT OF GOVERNMENT INTEREST

The United States' Army has certain rights in this invention.

### III. FIELD OF THE INVENTION

The present invention relates to medical diagnostic tools. More particularly, the present invention relates to a wearable device for assessing the viability of a tissue region.

### IV. BACKGROUND OF THE INVENTION

Medical diagnostic tools include processes and/or devices that assist people and/or other processes and/or devices in the assessment of medical conditions. One type of medical condition in which medical diagnostic tools prove particularly useful involves determining the viability of a tissue region. This may be the case, for example, in medical procedures involving skin grafting and/or burns to the skin. Presently, one approach to determine the depth of a burn is using laser Doppler perfusion imaging that can help look at blood flow in the skin, thereby assisting in making burn depth determinations.

In addition, certain medical devices need to be shielded from non-excitation light, or significant noise will be introduced, e.g., the device must be used in a dark room. As a result, the environments in which the devices may be used are limited.

Furthermore, such diagnostic tools are large, bulky instruments with limited mobility. In addition, typically medical professionals must use their hands to operate such devices, thereby interrupting or limiting the performance of medical procedures performed by hand. These systems can sometimes be mounted in the operating room, but would need to be positioned accordingly to evaluate the field of interest on the patient being examined. In the battlefield, using a mount for such devices is impractical and/or inconvenient.

Many diagnostic tools are not capable of capturing live streaming video, but can only capture single images taken at definite intervals such as the laser Doppler perfusion imaging system mentioned above. These systems also do not have the ability to determine the distance between the equipment being used and the patient even though the distance needs to be standardized if you are measuring light intensity and want to make valid comparisons among different subjects.

Accordingly, a need exists for a diagnostic tool which may be operated hands-free, and in various environments, for assessing the viability of a tissue region. Additionally, a need exists for a diagnostic tool capable of obtaining images at a standard distance for comparison purposes and analysis.

### V. BRIEF SUMMARY OF THE INVENTION

The present invention overcomes the limitations of the prior art as briefly described above, by providing a wearable device including night vision goggles that are responsive to a contrast agent. The present invention further overcomes the limitations of the prior art by providing a device that can provide a standardized distance-to-subject measurement.

An aspect of the present invention is directed to a method including delivering a contrast agent proximate to a tissue region and acquiring an image of the tissue region with a wearable device. The wearable device is responsive to the contrast agent. The severity of a burn (or viability of the tissue) is assessed in response to the acquired image.

More particularly, the method includes activating a light source of the wearable device, wherein the light source is configured to cause the contrast agent to fluoresce or absorb light. The acquired image may be transmitted to various devices. In addition, acquiring an image may include determining a distance to the tissue region via transmitted energy. The severity of a burn, for example, may be determined by assessing differences in contrasts of areas of the tissue region. Furthermore, the wearable device may include head mounting gear.

Another aspect of the present invention is directed to a wearable device for acquiring a contrast image of a tissue region of a patient including means for acquiring an image of the tissue region hands-free, and means for assessing a severity of a burn in response to the acquired image based on differing contrasts of the acquired image.

More particularly, the wearable device further comprises means for causing a contrast agent to fluoresce. The means for causing the contrast agent to fluoresce may be detachable from the wearable device. In addition, the device may include means for activating a light source of the wearable device, wherein the light source is configured to cause a contrast agent to fluoresce or absorb the light. The device may also include means for optically filtering the image to include pixels illuminated at frequencies about a wavelength at which a contrast agent fluoresces or absorbs.

Still another aspect of the present invention is directed to a device for gathering image information about a region of tissue that has been exposed to a contrast agent. The device includes night vision goggles, and an excitation source that generates light of a wavelength to activate the contrast agent. The excitation source is attached to the night vision goggles and is capable of directing light to a target. The device also includes a filter attached to the night vision goggles, wherein the filter passes light sufficient to form an image of the region of tissue, and wherein the image may be assessed to determine the viability of the region of tissue.

Another aspect of the present invention is related to a system including the device of the present invention and a computational device configured to communicate with the device. The computational device may be configured to assess the viability of tissue or the severity of a burn in a tissue region.

Yet another aspect of the present invention is directed to a kit to modify night vision goggles comprising a detachable unit, wherein the detachable unit includes dual diodes for determining the distance to a tissue region and an excitation source. The kit also includes a filter for passing wavelengths around at which the contrast agent fluoresces. The kit may include a body mounting device to allow the night vision goggles to be worn. More particularly, the kit may include a charge coupled diode camera, a switch for activating the excitation source, and/or means for transmitting the acquired image.

In one or more various implementations, related systems include but are not limited to circuitry and/or programming for effecting the foregoing-referenced method embodiments; the circuitry and/or programming can be virtually any combination of hardware, software, and/or firmware configured to effect the foregoing-referenced method embodiments depending upon the design choices of the system designer.

The accompanying figures show illustrative embodiments of the invention from which these and other of the objectives, novel features and advantages will be readily apparent.

### VI. BRIEF DESCRIPTION OF THE DRAWINGS

FIG. 1 illustrates a health care provider wearing a device of the present invention.

FIG. 2 depicts a health care provider wearing a device according to at least one embodiment of the present invention.

FIG. 3 illustrates an embodiment with distance determination means forming a part of a device of the present invention.

FIG. 4 depicts a perspective view of an embodiment of the present invention.

FIG. 5 illustrates a perspective view of an embodiment of the present invention.

FIG. 6 depicts an isolated view of a burned tissue region.

FIGS. 7A and 7B illustrate high-level flowcharts of a process according to at least one embodiment of the invention.

FIGS. 8A through 8F depict images acquired according to an embodiment of the present invention.

FIG. 9 depicts a conventional data processing system in which portions of the illustrative embodiments of the devices and/or processes described herein may be implemented to form an example of a computational system.

The use of the same symbols in different drawings typically indicates similar or identical items.

### VII. DETAILED DESCRIPTION OF THE INVENTION

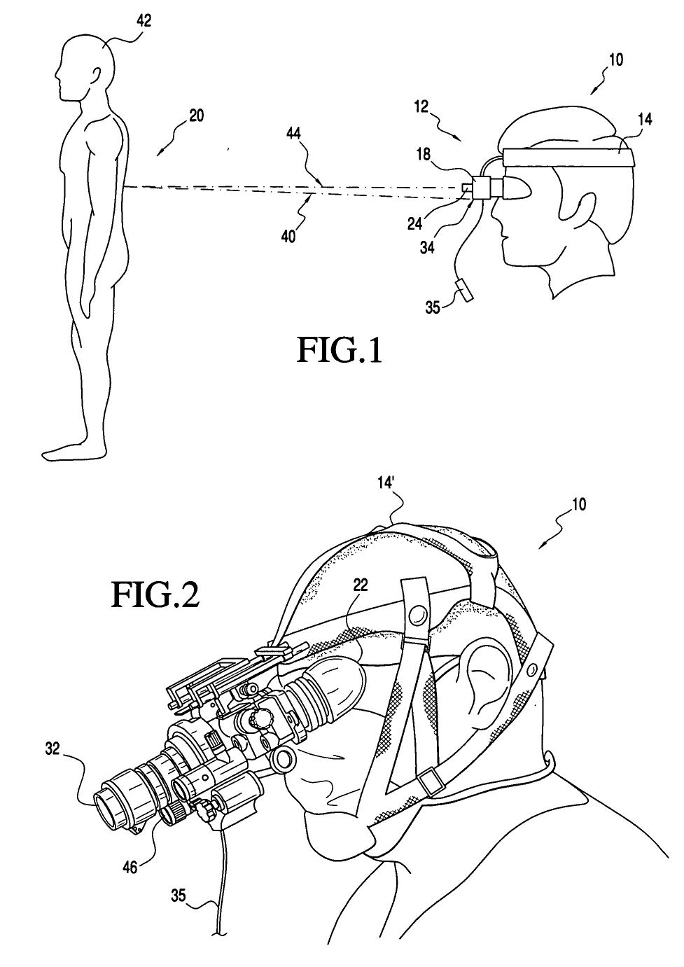

With reference to the figures, and with reference now to FIG. 1, shown is health care provider 10 using wearable device 12. The wearable device 12 may include a body mounting device (or gear) 14 (means for wearing) that allows the device to be worn on any part of the body for hands-free operation including, but not limited to the head, shoulder, chest or waist. For example, the body mounting device 14 may be a head mounting device as shown in FIGS. 1(14) and 2(14′). In other exemplary embodiments, the device 12 does not include mounting device 14. Thus, a surgeon may operate with the device 12 mounted on his/her head, observing tissue perfusion in areas of interest (tissue region 20) to make a decision on whether or not a particular area, for example, needs to be deeply excised or regrafted. The tissue region 20 may be any in vivo or in vitro tissue region including, but not limited to cells, growths, tumors, other tissues of interest, or tissues of a human or animal body organ system such as integument (skin), musculoskeletal, nervous, endocrine, circulatory, respiratory, urinary, gastrointestinal, reproductive, and immunological. Applications include assessing the severity of burns, assessing perfusion of skin or other tissue grafts, identifying the bile duct, or tumor vascularization.

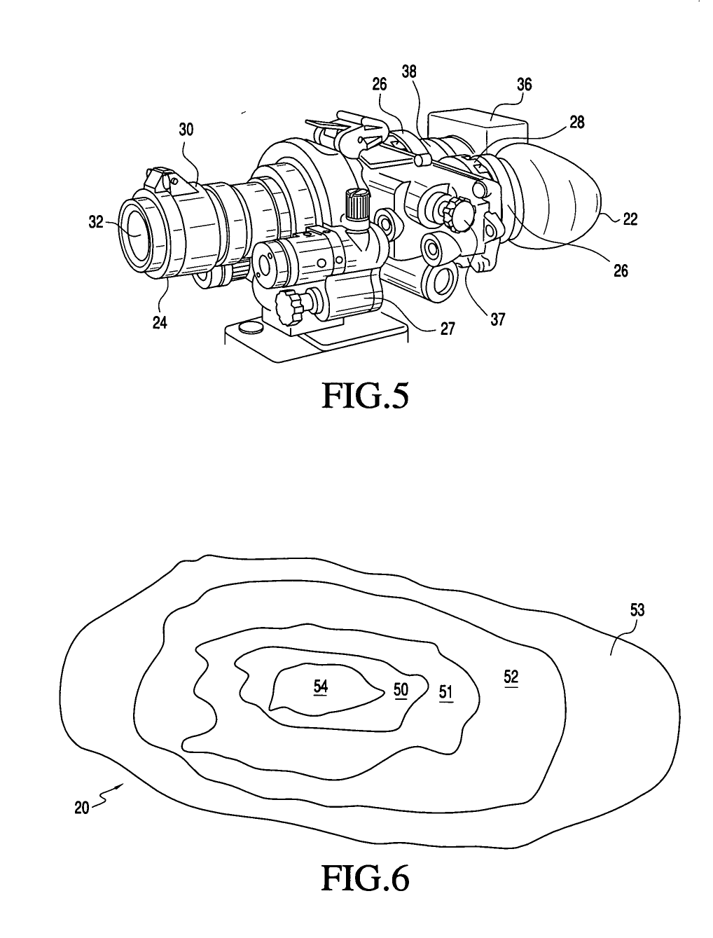

The wearable device 12 includes a means for acquiring an image 18 of the tissue region 20. The means for acquiring an image 18 of the tissue region 20 is preferably night vision goggles which may be battery operated. Appropriate night vision goggles for use in the present invention include night vision goggles part no. AN/PVS-7, available from Night Vision Equipment Company located in Allentown, Pa. As shown in FIGS. 4 and 5, the night vision goggles include one or two eyepieces 22, a focusable lens 24, an optical fluorescence filter 32, an interpupillary adjustment control 28, and diopter adjustment controls 26. The focusable lens 24 may be a single tube objective lens that includes a focusing ring 30 (which may be marked in one embodiment to quickly and accurately focus for a set distance) and the filter 32. FIG. 4 illustrates the filter 32 shown in an “up” position, which allows the night vision goggles to perform as regular night vision goggles. FIG. 5 illustrates an exemplary two-position switch 27 for turning on the red laser diodes 46 and excitation source 34 in at least one embodiment. One of ordinary skill in the art will appreciate that night vision goggles can come in a variety of configurations.

The night vision goggles also include an excitation source 34 (means for causing a contrast agent to fluoresce or absorb) having an optical filter. The excitation source 34 preferably includes an aperture (not shown) for increasing or decreasing the excitation laser emitted from the excitation source. The device 12 preferably includes an activating means (or laser activation control) 35 such as a switch or other actuating member which may be operated by hand or by foot (which may have a wired or wireless connection), for example, to control the intensity of the excitation source 34 once the excitation source has been turned on. Examples of a laser activation control 35 suitable for use in the present invention include a toggle, switch, a push pad, a pedal, and similar mechanisms that can be used by an operator to preferably cause the laser intensity to increase once activated and decrease when inactivated (or in the case of a switch turned on and turned off).

A power switch 37 is also included for turning power to the device 12 on and off. An LED may be provided to indicate when the power is on or off on the entire device and/or individual components. The attached light source(s) may in one embodiment have their own on/off switches.

The night vision goggles may also include an optional charge coupled diode (CCD) chipset camera 36 having an adapter 38 for export of live and/or still images. In one implementation, CCD camera 36 fits into an eyepiece 22 of the night vision goggles, leaving the other eyepiece available for concurrent viewing by a user's eye as illustrated in FIG. 5. In another implementation, CCD camera 36 is mounted in an optical path coincident with the eyepiece 22 and uses optics and/or electronics to capture the image transmitted to the eyepiece.

Referring back to FIG. 1, the excitation light is transmitted from excitation source 34, along path 40, to impinge upon tissue region 20, for example, that contains one or more skin burns of burn victim 42. In one implementation, the excitation source 34 is a laser diode. In one implementation, excitation source 34 has a center frequency of 780 nanometers (nm) and a bandwidth of 10 nm, for example, when the contrast agent includes indocyanine green dye. However, the exact center frequency and bandwidth of excitation source 34 is a design choice dependent upon one or more of desired noise immunity factors, fluorescent or absorption properties of the contrast agent (or is keyed to the wavelength of the contrast agent), and reception properties of the device 12. For example, if the contrast agent is a heptamethine cyanine, the excitation source 34 may have a center frequency of 760 to 800 nm depending upon the heptamethine cyanine used. In the situation where the contrast agent is light absorbing, then the filter would allow wavelengths of light that would be reflected by the surrounding tissue with less or no contrast agent present.

In one implementation (not shown), a filter is placed in front of the excitation source 34 to limit the spectrum of the supplied light, which will allow use of a larger bandwith filter on the received pathway. The contrast agent may be any material that fluoresces between about 700 and about 900 nm (more particularly, in a range of 790 to 840 nm), or absorbs light. Examples of near infrared contrast agents appear in the following article, which are hereby incorporated by reference for examples of contrast agents: Frangioni J V, Review of near-infrared fluorescence imaging, Curr Opinion Chem Biol 2003; 7: 626-634. In one implementation, the contrast agent (previously delivered proximate to the tissue region, as explained below) that has leaked from the capillaries perfusing a burn in region 20 fluoresces in response to the excitation light, and light arising from that florescence follows path 44 to objective lens 24. The working distance between health care provider 10 and burn victim 42 or the tissue region 20 typically ranges between six inches to five feet (6″ to 5′) for use during diagnosis, surgery, or treatment. The distance range may be tailored to coincide with the working arm length of the user such as a surgeon. An alternative possibility for the range for the working distance is between two feet to eight feet (2′-8′). In one implementation, the filter 32 of objective lens 24 has a center frequency of 840 nm and a bandwidth of 10 nm. However, the exact center frequency and bandwidth of the optical fluorescence filter is a design choice dependent upon one or more of noise immunity factors, fluorescent or absorbent properties of the contrast agent, reception properties of the device 12, and transmission characteristics of excitation source 34. The filter 32 optically filters the acquired image to produce pixels illuminated at frequencies about a wavelength at which the contrast agent fluoresces or absorbs. The excitation source 34 and lens 24 are preferably a matched set in that they work with a particular contrast agent.

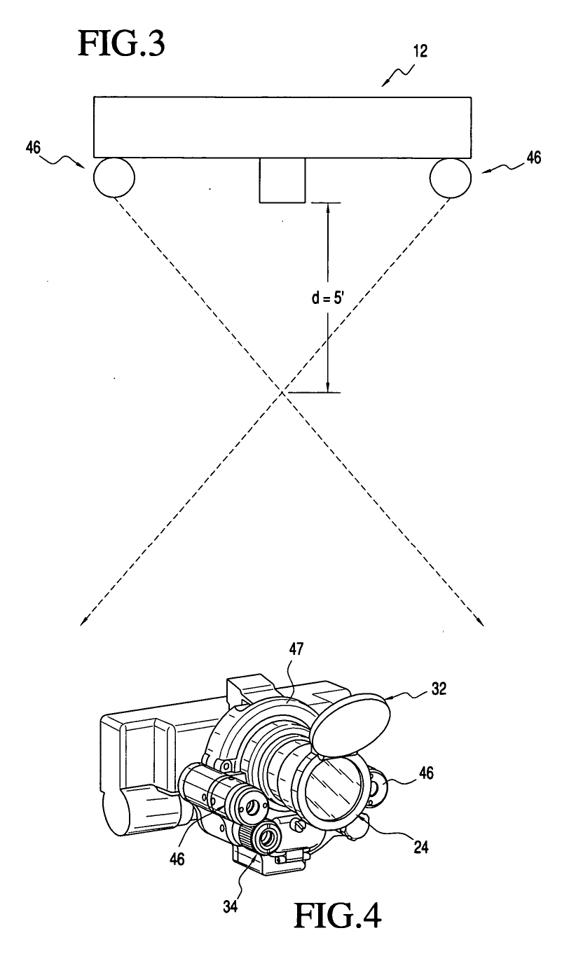

With reference now to FIG. 3, illustrated are dual adjustable focusing red laser diodes (or light emitting diodes (LEDs) that are adapted to maintain a focused beam over a distance) 46 (means for determining a distance to the tissue region) forming a part of wearable device 12. Dual diodes 46 are oriented and/or focused such that they cross at a predetermined distance (e.g., 5′) from device 12. Dual diodes 46 allow distances between different viewing events to be standardized if such is needed or desired. In one implementation, dual diodes 46 are powered from a waist mounted battery pack. In one embodiment, dual diodes 46 use conventional focusing and/or aiming mechanisms. In one embodiment, the dual diodes 46 and the excitation source 34 are removably mounted on the wearable device 12, and can then be remounted to that wearable device 12 or attached to another wearable device. An example of one way to accomplish this is a detachable unit 47 illustrated in, for example, FIG. 4.

Referring now to FIG. 6, depicted is an isolated view of tissue region 20 that contains one or more skin burns. Tissue region 20 contains burned region 50, moderately burned region 51 and slightly burned region 52 surrounded by a region of unburned skin 53. The skin burns become progressively more serious toward the interior of tissue region 20. The outermost skin 53 is undamaged. The innermost skin region 54 is charred. It should be noted that the device 12 of the present invention may be used to assess tissue burns, including the depth of a burn, caused by any source such as thermal, chemical, electrical, UV, biologic, etc.

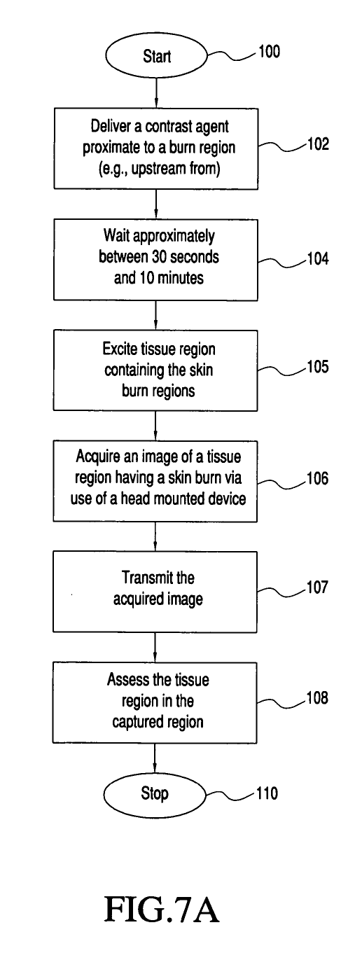

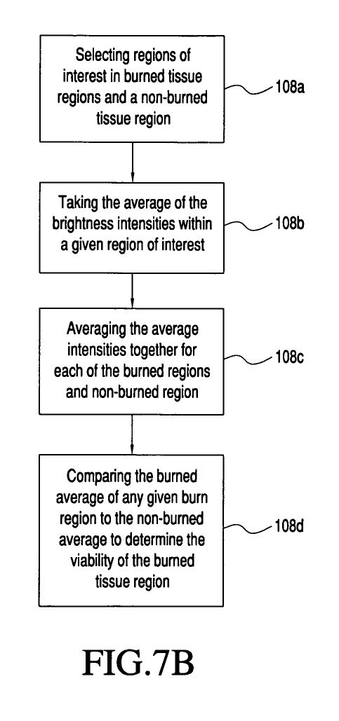

With reference now to FIG. 7A, illustrated is a high-level flowchart of a process described in the context of FIGS. 1-6. Method step 100 shows the start of the process. Method step 102 depicts delivering a contrast agent, such as indocyanine green dye, intravascularly proximate to a tissue region to be examined such as the burn tissue illustrated in FIG. 6. Delivering includes injecting, spraying, applying and other related methods. As used herein, intravascularly proximate means that the contrast agent is delivered such that the amount of contrast agent that ultimately reaches the tissue region 20 will provide diagnostically acceptable amounts of fluorescence. Typically, the contrast agent is injected “upstream” from the tissue region 20 such that, in an undamaged patient, the capillary system would carry the contrast agent into the tissue region. While injecting (e.g., via a hypodermic needle) is described for sake of illustration, the term injecting in method step 102 is meant to include virtually all ways in which the contrast agent may be introduced into skin capillaries. In various implementations, dosages of 0.2 milligram (mg), 0.5 mg, 1.0 mg, 2.0 mg, and 5.0 mg of indocyanine green dye per kilogram (kg) of animal body weight are injected by health care provider 10. Multiple boluses may be injected as needed. In instances in which the animal is a human and the contrast agent being used is indocyanine green dye, it is preferable to keep the total delivered dosage below 2.0 mg per kg.

Method step 104 shows that health care provider 10 waits for a period of time, which generally ranges between 30 seconds and 10 minutes until maximal contrast appears between the tissue of interest and surrounding (normal) tissue. In one implementation, a period of 5 minutes has proven advantageous. However, in some cases it may not be necessary for the health care provider to wait for a period of time.

Method step 105 depicts health care provider 10 exciting the tissue region 20 containing the skin burn regions 50, 51 and 52 with excitation source 34, where excitation source 34 generates light of a wavelength to activate the contrast agent. Activating the contrast agent may cause the contrast agent to fluoresce or absorb the excitation light. In one implementation of method step 105, the excitation is achieved by activating a laser diode (or source) that forms a part of the device 12. This method step may include the further step of firing the laser diode on demand.

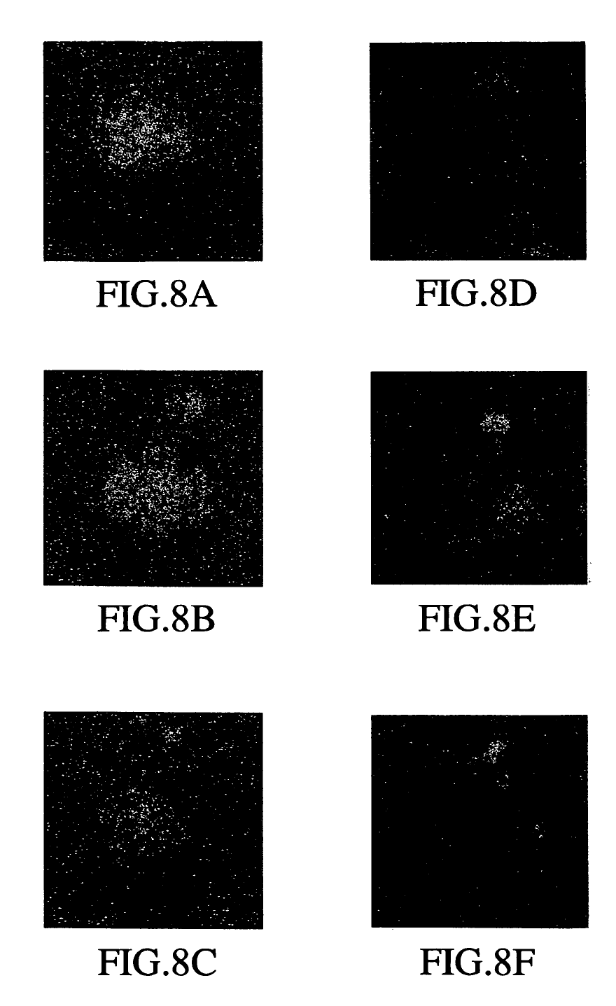

Method step 106 depicts acquiring an image of all or part of tissue region 20 containing the skin burns via use of wearable device 12. In one implementation of method step 106, the image is acquired with night vision goggles. In another implementation of method step 106, the image is filtered with an optical bandpass filter 32 affixed to night vision goggles, where the optical bandpass filter 32 has a passband that includes a wavelength at which the contrast agent fluoresces or absorbs. The acquired image may provide a large field for view, e.g., the entire arm, chest or back of a patient, or small fields of view. FIGS. 8A-8C illustrate sample acquired images taken about 24 hours (FIG. 8A), 48 hours (FIG. 8B), and 72 hours (FIG. 8C) after a superficial dermal (2nd degree) sulfur mustard burn was experimentally induced in a pig model where the contrast agent that was used is indocyanine green dye. FIGS. 8D-8F illustrate sample acquired images taken about 24 hours (FIG. 8D), 48 hours (FIG. 8E), and 72 hours (FIG. 8F) after a deep (third degree) sulfur mustard burn was experimentally induced in a pig model where the contrast agent that was used is indocyanine green dye.

Method step 107 illustrates transmitting the acquired image. In one implementation of method step 107, the acquired image is directly transmitted to health care provider 10 via the eyepieces 22. In another implementation of method step 107, the acquired image is captured by CCD chipset camera 36, and then transmitted to a computer for display on a monitor, storage, analysis, and/or transmission such as in telemedicine (e.g., transmission to a remote site via satellite). In another implementation of method step 107, health care provider 10 or a technician can encircle a burn region 20 in the acquired image using a mouse, joystick, or other device and then retransmit the image to another device (e.g., another computer or a satellite). Thus, means for transmitting the acquired image may include eyepieces 22 and/or a CCD camera 36.

Method step 108 shows assessing the severity of the burn in the captured image. Images to be processed may be snapshots taken at specific points in time following delivery of the contrast agent, or individual frames grabbed from live streaming video captured from the CCD camera 36. In one implementation of method step 108, health care provider 10 subjectively assesses burn severity in response to varying contrasts, such as varying brightnesses, of the captured image of the burned tissue regions 50, 51 and 52 and the surrounding undamaged region 53. In another implementation of method step 108, the severity of burns or viability of tissue in the acquired image can be calculated by computerized image processing on the basis of pixel differences related to the response of undamaged, or unburned tissue. For example, assessing the severity of burns or viability of tissue in tissue region 20 may include the following exemplary steps illustrated in FIG. 7B. Method step 108a includes selecting multiple regions of interest (e.g., 5 per burn region) in each of the burned regions (or first area(s)) 50, 51, 52 and in the non-burned region (or healthy area or second area) 53 (e.g., 3 regions). Method step 108b includes taking the average of the brightness intensities of all pixels within a given region of interest. Method step 108c includes averaging the average intensities from all regions of interest within any single burned region together and from the non-burned area together, respectively. Method step 108d includes comparing the burned average of any given burned region to the non-burned average to determine the viability of the burned tissue. One of ordinary skill in the art will appreciate that this method can be used to assess other types of tissue besides burned tissue.

The response of the unburned or undamaged tissue can be either pre-stored or obtained in near real time from the patient. Examples of processes appear in the following described articles, which are hereby incorporated by reference in their entireties: Jerath M R, Schomacker K T, Sheridan R L, Nishioka N S, Burn Wound Assessment In Porcine Skin Using Indocyanine Green Fluorescence, J Trauma 1999 June; 46(6): 1085-8; Sheridan R L, Schomaker K T, Lucchina L C, Hurley J, Yin L M, Tompkins R G, Jerath M, Torri A, Greaves K W, Bua D P, Burn Depth Estimation By Use Of Indocyanine Green Fluorescence: Initial Human Trial, J Burn Care Rehabil 1995 November-December; 16(6):602-4; Still J M, Law E J, Klavuhn K G, Island T C, Holtz J Z., Diagnosis Of Burn Depth Using Laser-Induced Indocyanine Green Fluorescence: A Preliminary Clinical Trial, Burns 2001 June; 27(4):364-71; Schomacker K T, Torri A, Sandison D R, Sheridan R L, Nishioka N S, Biodistribution Of Indocyanine Green In A Porcine Burn Model: Light And Fluorescence Microscopy, J Trauma 1997 November; 43(5):813-9. In one implementation of method step 108, image processing in a computational device processes the brightnesses and/or contrasts of adjacent pixels and assesses the burn severity based on such processing, while in another implementation the processing is accomplished by components in wearable device 12. In addition to the foregoing described techniques, another implementation uses image processing techniques appearing in U.S. Pat. No. 5,074,306 to Green et al. (24 Dec. 2001), such patent application hereby incorporated by reference in its entirety.

Method step 110 shows the end of the process.

It should be understood that the above described method and device of the present invention is not limited to determining the severity of burns in a tissue region 20, but may also be used to assess the viability of any tissue region, including but not limited to, determining the blood flow through a tissue region for procedures involving skin grafts, skin flaps, or intestinal surgery. The method and device can also be used in vivo or in vitro to analyze growths, tumors, or other tissues of interest where the contrast agent is attached to, for example, antibodies or ligands that will attach to specific sites (determined by the specificity of the antibody) in or on the growths, tumors, or other tissue of interest, if present, and after application any excess contrast agent is washed away (or otherwise removed) prior to viewing the tissue region. The contrast agent instead of being injected or sprayed may be applied in other ways to the tissue known to those of ordinary skill in the art.

While the concepts involving burn assessment are complex, in general the underlying idea of the assessment can be understood as follows: for slight, moderate, and badly burned skin, subsequent to a contrast agent being injected upstream of the burned tissue region 20, the capillary system in the skin will leak. The varying burns will have varying amounts of leakage, and hence varying fluorescent brightness levels. For instance, slightly burned areas 52 will have good blood flow into the area and leaky vessels; therefore, after injection of the contrast agent such areas will appear much brighter than undamaged skin 53. For badly burned areas 50, blood flow is poor or absent, and very little contrast agent will enter the area; therefore, after injection of a contrast agent such areas will appear darker than undamaged skin 53. For moderately burned areas 51, the blood flow into the area and the leakiness of vessels will be intermediate between slight and badly burned. Various embodiments of the subject matter of the present application make it possible to localize where the viable skin ends and the damaged beyond repair skin begins. For example, the device 12 and methods of the present invention may be used to determine the blood flow through an intestine and other organs of the body.

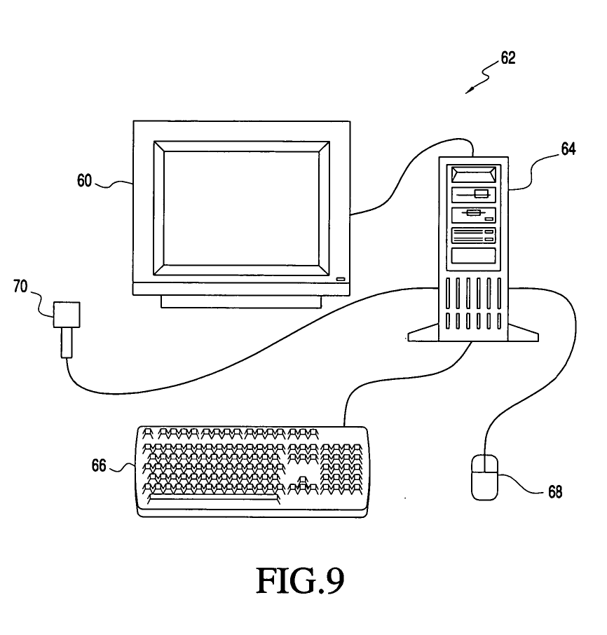

As described above, the optional CCD camera 36 may include a transmitter or transmitting means that exchanges signals with a computational device (or storing means which may instead be resident on the CCD camera) as illustrated in FIG. 9. As noted herein, the computational device can store and/or process and/or further transmit the data. In one embodiment, the computational device displays the burn assessment on a video display device 60. In another embodiment, the computational device transmits data back to the wearable device 12 which then transmits the tissue assessment information into the eyepiece(s) 22 so that health care provider 10 has an objective measure of tissue assessment.

Those having ordinary skill in the art will recognize that the state of the art has progressed to the point where there is little distinction left between hardware and software implementations of aspects of systems; the use of hardware or software is generally (but not always, in that in certain contexts the choice between hardware and software can become significant) a design choice representing cost vs. efficiency tradeoffs. Those having ordinary skill in the art will appreciate that there are various computational and/or other devices by which aspects of processes and/or systems described herein can be effected (e.g., hardware, software, and/or firmware), and that the preferred vehicle will vary with the context in which the processes and/or systems are deployed. For example, if an implementer determines that speed and accuracy are paramount, the implementer may opt for a hardware and/or firmware vehicle; alternatively, if flexibility is paramount, the implementer may opt for a solely software implementation; or, yet again alternatively, the implementer may opt for some combination of hardware, software, and/or firmware. Hence, there are several possible vehicles by which aspects of the processes described herein may be effected, none of which is inherently superior to the other in that any vehicle to be utilized is a choice dependent upon the context in which the vehicle will be deployed and the specific concerns (e.g., speed, flexibility, or predictability) of the implementer, any of which may vary.

The foregoing detailed description has set forth various embodiments of the devices and/or processes via the use of block diagrams, flowcharts, and examples. Insofar as such block diagrams, flowcharts, and examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In one embodiment, the present invention may be implemented via Application Specific Integrated Circuits (ASICs). However, those skilled in the art will recognize that the embodiments disclosed herein, in whole or in part, can be equivalently implemented in standard Integrated Circuits, as one or more computer programs running on one or more computers (e.g., as one or more programs running on one or more computer systems), as one or more programs running on one or more controllers (e.g., microcontrollers) as one or more programs running on one or more processors (e.g., microprocessors), as firmware, or as virtually any combination thereof, and that designing the circuitry and/or writing the code for the software and or firmware would be well within the skill of one of ordinary skill in the art in light of this disclosure. In addition, those skilled in the art will appreciate that the mechanisms of the present invention are capable of being distributed as a program product in a variety of forms, and that an illustrative embodiment of the present invention applies equally regardless of the particular type of signal bearing media used to actually carry out the distribution. Examples of signal bearing media include, but are not limited to, the following: recordable type media such as floppy disks, hard disk drives, CD ROMs, digital tape, and computer memory; and transmission type media such as digital and analogue communication links using TDM or IP based communication links (e.g., packet links) or carrier signals.

In a general sense, those skilled in the art will recognize that the various embodiments described herein which can be implemented, individually and/or collectively, by a wide range of hardware, software, firmware, or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), and electrical circuitry forming a communications device (e.g., a modem, communications switch, or optical-electrical equipment).

Those skilled in the art will recognize that it is common within the art to describe devices and/or processes in the fashion set forth herein, and thereafter use standard engineering practices to integrate such described devices and/or processes into systems. That is, the devices and/or processes described herein can be integrated into a system via a reasonable amount of experimentation. FIG. 9 shows an example representation of a data processing system into which at least a part of the herein described devices and/or processes may be integrated with a reasonable amount of experimentation.

With reference now to FIG. 9, depicted is a conventional data processing system in which portions of the illustrative embodiments of the devices and/or processes described herein may be implemented to form an example of a computational system. It should be noted that graphical user interface systems (e.g., Microsoft Windows operating systems) and methods may be utilized with the data processing system depicted in FIG. 9. Data processing system 62 is depicted which includes system unit housing 64, video display device 60, keyboard 66, mouse 68, and microphone 70. Data processing system 62 may be implemented utilizing any suitable commercially available computer system. In one embodiment, data processing system 62 is running a computer program and is linked with the wearable device 12 via wireless communications equipment (not shown) and other facilities via satellite communication equipment (not shown).

The foregoing described embodiments depict different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality. For example, any of the components of the present invention described above may be combined to form a kit to modify night vision goggles for the purpose of assessing a tissue region 20.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. For instance, although a head mounted device has been shown and described herein, it is to be understood that the head mounted device is merely a specific example of more general wearable devices. Wearable devices are devices that may be worn by a person. Other examples of a wearable devices are vest devices and backpack devices. Furthermore, it is to be understood that the invention is solely defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

So far as the inventors are aware, related art systems need to be shielded from non-excitation light, or significant noise will be introduced (e.g., should be operated in a dark room). Some implementations of the subject matter disclosed herein can be used in most lighting conditions without the need for additional shielding material due to the use of the fluorescent dye and the fluorescent optical filters. In some instances this is achieved by increasing the strength of the excitation source, decreasing the bandwidth of the optical filter, and/or increasing the dosage of the contrast agent. In other implementations the fluorescence is outside of the range where significant natural and/or artificial electromagnetic radiation sources are likely to be found.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A method comprising:
delivering a contrast agent proximate to a tissue region;
acquiring an image of the tissue region with a wearable device, the wearable device includes night vision goggles and is responsive to a contrast agent; and
assessing a severity of a burn in response to the acquired image.

2. The method of claim 1, further comprising activating a light source of the wearable device, wherein the light source is configured to cause the contrast agent to fluoresce.

3. The method of claim 1, further comprising wearing the device using head mounting gear.

4. The method of claim 1, wherein acquiring an image includes determining a distance to the skin region via transmitted energy.

5. The method of claim 4, wherein assessing a severity of a burn comprises:
assessing differences in contrasts of areas of the tissue region.

6. The method of claim 4, further comprising transmitting the acquired image.

7. The method of claim 4, wherein assessing a severity of a burn includes:
selecting regions of interest in a first area of burn tissue and a second area of non-burned tissue in the tissue region;
averaging a brightness intensity of all pixels within each region of interest; and
calculating a ratio between the average intensity of a first area versus a second area in the tissue region.

8. A computer data signal embodied in a carrier wave readable by a computing system and encoding a computer program of instructions for executing a computer process performing the assessing steps recited in claim 7.

9. A computer-readable medium having computer-executable instructions for the assessing steps recited in claim 7.

10. A wearable device for acquiring a contrast image of a tissue region of a patient comprising:
means for acquiring an image of the tissue region hands-free; and
means for assessing a severity of a burn in response to the acquired image based on differing contrasts of the acquired image.

11. The device of claim 10, further comprising means for causing a contrast agent to fluoresce.

12. The device of claim 11, wherein the means for causing the contrast agent to fluoresce is detachable from the wearable device.

13. The device of claim 10, wherein means for acquiring an image comprises:
means for determining a distance to the tissue region.

14. The device of claim 13, wherein means for acquiring comprises:
means for optically filtering the image to include pixels illuminated at frequencies about a wavelength at which the contrast agent fluoresces.

15. The device of claim 13, further comprising:
means for transmitting the acquired image.

16. The device of claim 13, wherein means for acquiring an image comprises:
means for storing the image.

17. The device of claim 13, further comprising:
means for activating a light source of the wearable device, wherein the light source is configured to cause a contrast agent to fluoresce.

18. The device of claim 10, further comprising:
means for wearing the device on a head, shoulder, chest or waist.

19. A device for gathering image information about a region of tissue that has been exposed to a contrast agent comprising:
night vision goggles,
an excitation source that generates light of a wavelength to activate the contrast agent, said excitation source is attached to the night vision goggles and is capable of directing light to a target, and
a filter attached to the night vision goggles, said filter passing light sufficient to form an image of the region of tissue, wherein the image may be assessed to determine the viability of the region of tissue.

20. A system comprising:
the device of claim 19, and
a computational device configured to communicate with the device.

21. A system comprising:
the device of claim 19, and
a computational device configured to assess the burn severity.

22. The device of claim 19, wherein the excitation source includes a laser source.

23. The device of claim 19, wherein the excitation source is detachable from the device.

24. The device of claim 19, further comprising a distance determination device attached to the night vision goggles.

25. The device of claim 24, wherein the distance determination device includes dual diodes.

26. The device of claim 24, further comprising a means for activating the excitation source.

27. The device of claim 24, further comprising a Charge Coupled Diode camera.

28. The device of claim 19, further comprising a body mounting device.

29. The device of claim 28, further comprising:
a distance determination device,
a switch connected to the excitation source, and
a Charge Coupled Diode camera.

30. A kit to modify night vision goggles comprising:
a detachable unit comprising:
dual diodes for determining the distance to a tissue region, and
an excitation source; and
a filter for passing wavelengths around at which the contrasting agent fluoresces, and wherein an image is formed based on the passed wavelengths that can be assessed to determine the viability of the tissue region.

31. The kit of claim 30, further comprising:
a body mounting device to allow the night vision goggles to be worn.

32. The kit of claim 30, wherein the body mounting device is a head mounting device.

33. The kit of claim 30, further comprising:
a switch in communication with the excitation source.

34. The kit of claim 30, further comprising:
a Charge Coupled Diode camera for acquiring the image.

35. The kit of claim 30, further comprising means for transmitting the image.

36. The kit of claim 35, further comprising:
a body mounting device to allow the night vision goggles to be worn,
a switch for activating the excitation source, and
a Charge Coupled Diode camera for acquiring the image.

37. The kit of claim 35, wherein said dual diodes are red laser diodes.
